(19) United States
(12) Patent Application Publication
(10) Pub. No.: US 2024/0016616 A1
(43) Pub. Date: Jan. 18, 2024

(54) INFORMATION PROCESSING APPARATUS, INFORMATION PROCESSING METHOD, AND RECORDING MEDIUM

(71) Applicant: KYOCERA Corporation, Kyoto-shi (JP)

(72) Inventors: Shota NAKASUJI, Otsu (JP); Takahiro TAKAYA, Uji (JP)

(73) Assignee: KYOCERA Corporation, Kyoto-shi (JP)

(21) Appl. No.: 18/477,034

(22) Filed: Sep. 28, 2023

Related U.S. Application Data
(63) Continuation of application No. 16/387,607, filed on Apr. 18, 2019.

(30) Foreign Application Priority Data
Apr. 25, 2018 (JP) 2018-084121

(51) Int. Cl. A61F 2/30; A61F 2/34; A61F 2/36

(52) U.S. Cl. CPC A61F 2/30942; A61F 2/34; A61F 2/3662; A61F 2002/30952; A61F 2002/365; A61F 2002/4658

(57) ABSTRACT

An information processing apparatus includes a controller that determines the height at which to fix a cup of an artificial hip joint in a patient in accordance with the femoral head center on the contralateral side opposite from the affected hip joint of the patient, a teardrop lower edge of a hip joint of the patient, and the CE angle of the affected hip joint of the patient.

### CROSS-REFERENCE TO RELATED APPLICATION

This application claims priority to and the benefit of U.S. patent application Ser. No. 16/387,607, filed Apr. 18, 2019 and Japanese Patent Application No. 2018-084121, filed Apr. 25, 2018, the entire contents of which are incorporated herein by reference.

### TECHNICAL FIELD

The present disclosure relates to an information processing apparatus, an information processing method, and a recording medium.

### BACKGROUND

Artificial joint replacement surgery to replace a deformed joint with an artificial joint is widely performed. Apparatuses and the like used to support a preoperative plan for artificial joint replacement surgery are known.

### SUMMARY

Known apparatuses and the like to support a preoperative plan for artificial joint replacement surgery have room for improvement.

The present disclosure relates to an improved information processing apparatus, information processing method, and recording medium.

An information processing apparatus according to an embodiment includes a controller configured to determine a height at which to fix a cup of an artificial hip joint in a patient in accordance with a femoral head center on a contralateral side opposite from an affected hip joint of the patient, a teardrop lower edge of a hip joint of the patient, and a CE angle of the affected hip joint of the patient.

An information processing method according to an embodiment is executed by an information processing apparatus including a controller. The information processing method includes determining a height at which to fix a cup of an artificial hip joint in a patient in accordance with a femoral head center on a contralateral side opposite from an affected hip joint of the patient, a teardrop lower edge of a hip joint of the patient, and a CE angle of the affected hip joint of the patient.

A non-transitory computer-readable recording medium according to an aspect stores computer program instructions, that when executed by a computer, perform steps including determining a height at which to fix a cup of an artificial hip joint in a patient in accordance with a femoral head center on a contralateral side opposite from an affected hip joint of the patient, a teardrop lower edge of a hip joint of the patient, and a CE angle of the affected hip joint of the patient.

### BRIEF DESCRIPTION OF THE DRAWINGS

In the accompanying drawings:

FIG. 1 is a functional block diagram illustrating an example configuration of an information processing apparatus according to an embodiment;

FIG. 2 illustrates an example of an artificial hip joint;

FIG. 3 is a flowchart illustrating an example of processing, executed by the information processing apparatus of FIG. 1, for a preoperative plan;

FIG. 4 illustrates an example of information pertaining to a preoperative plan for artificial hip joint replacement surgery;

FIG. 5 illustrates leg length discrepancy;

FIG. 6 illustrates the CE angle and the outward opening angle in artificial hip joint replacement surgery;

FIG. 7 illustrates the CE angle in artificial hip joint replacement surgery; and

FIG. 8 is a flowchart illustrating an example of processing, executed by the information processing apparatus of FIG. 1, to determine the cup height.

### DETAILED DESCRIPTION

Embodiments are described below in detail with reference to the drawings.

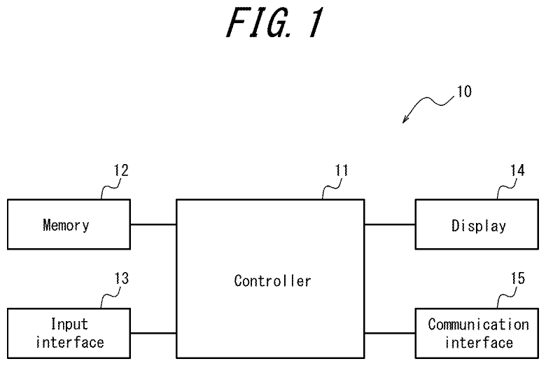

FIG. 1 is a functional block diagram illustrating an example configuration of an information processing apparatus 10 according to an embodiment. The information processing apparatus 10 can, for example, be constituted by a computer or the like. The information processing apparatus 10 is used in artificial hip joint replacement surgery to replace a patient's deformed hip joint with an artificial hip joint (implant). The information processing apparatus 10 creates a preoperative plan in accordance with inputted patient information and the like. For example, the information processing apparatus 10 determines the arrangement of the implant and the type of implant as the preoperative plan. The type of implant may, for example, include the size, shape, and the like of the implant.

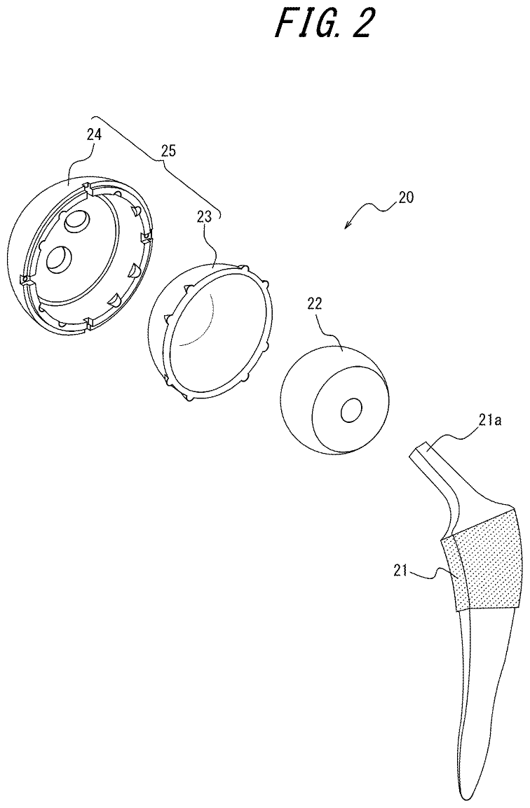

FIG. 2 illustrates an example of an artificial hip joint. As illustrated in FIG. 2, the artificial hip joint 20 includes a stem 21, a femoral head ball 22, a liner 23, and a shell 24. The stem 21 is fixed to the patient's femur. The stem 21 is, for example, a metal member. The femoral head ball 22 is fixed by mating to a tip 21a of the stem 21 and functions as the femoral head of the artificial joint. The liner 23 and the shell 24 constitute a cup 25, which is fixed to the acetabulum and which slidably houses the femoral head ball 22. The liner 23 is a resin member, such as polyethylene, with a substantially hemispherical shape that has a space capable of housing the femoral head ball 22 therein. The shell 24 is a member with a substantially hemispherical shape that houses the liner 23 therein. In the present disclosure, the open side of the cup 25 is referred to as the front of the cup 25. Accordingly, the cup 25 has a half-moon shape in a side view. A line that traverses the central portion of the circular opening and is perpendicular to the surface on which the opening is formed is referred to as the central axis of the cup 25.

In the present embodiment, the information processing apparatus 10 determines the position and angle at which to fix the cup 25 on the patient's acetabulum and the size of the cup 25 as the preoperative plan. The position at which to fix the cup 25 includes the height at which to fix the cup 25 on the acetabulum and the depth at which to embed the cup 25 in the acetabulum.

Referring again to FIG. 1, the information processing apparatus 10 illustrated in FIG. 1 includes a controller 11, a memory 12, an input interface 13, a display 14, and a communication interface 15.

The controller 11 is a processor that controls and manages the information processing apparatus 10 overall, starting with the functional blocks of the information processing apparatus 10. The controller 11 is a processor, such as a central processing unit (CPU), that executes a program with prescribed control procedures. Such a program may, for example, be stored in the memory 12 or on an external storage medium or the like connected to the information processing apparatus 10.

In the present embodiment, the controller 11 determines the position and angle at which to fix the cup 25 on the patient's acetabulum and the size of the cup 25 as the preoperative plan. The processing executed by the controller 11 for the preoperative plan is described in detail below.

The memory 12 includes a variety of memory devices and records various information depending on the intended use. For example, the memory 12 stores data necessary for operation of the controller 11. The memory 12 may include a device such as random access memory (RAM) that functions as a working memory. The memory 12 may, for example, store information related to artificial hip joints that a doctor can use in artificial hip joint replacement surgery. For example, the memory 12 may store the size, shape, and the like of artificial hip joints that a doctor can use. The memory 12 stores, for example, a predetermined algorithm that the controller 11 uses to create the preoperative plan.

The input interface 13 receives operation input from a user of the information processing apparatus 10 and may include operation buttons (operation keys), for example. The input interface 13 may, for example, include a keyboard and a mouse. The input interface 13 may be constituted by a touch panel, and operation input from the user may be received by a portion of a display device displaying operation keys that receive operation input from the user.

The display 14 is a display device constituted by a well-known display, such as a liquid crystal display (LCD), an organic electro-luminescence display (OELD), or an inorganic electro-luminescence display (IELD). The display 14 displays various information. For example, the display 14 displays the preoperative plan created by the information processing apparatus 10. The display 14 may, for example, display information related to the position and angle at which to fix the cup 25 and the size of the cup 25, which were determined as the preoperative plan.

The communication interface 15 can, for example, communicate with an external device using a network that is wireless, wired, or a combination of wireless and wired. The communication interface 15 can, for example, transmit and receive information over the network. The communication interface 15 communicates using a communication method of a wireless communication standard, for example. Examples of wireless communication standards include Worldwide Interoperability for Microwave Access (WiMAX), IEEE802.11, Bluetooth® (Bluetooth is a registered trademark in Japan, other countries, or both), Infrared Data Association (IrDA), Near Field Communication (NFC), communication standards conforming to ARIB STD-T109 (for example, Rev. 1.2), and communication standards conforming to ITS FORUM RC-010. The communication interface 15 may support one or more of the communication standards listed above as examples. To support a plurality of communication standards, the communication interface 15 may include a plurality of communication modules corresponding to respective wireless communication standards. For example, the information processing apparatus 10 can transmit information related to the created preoperative plan to another external device via the communication interface 15.

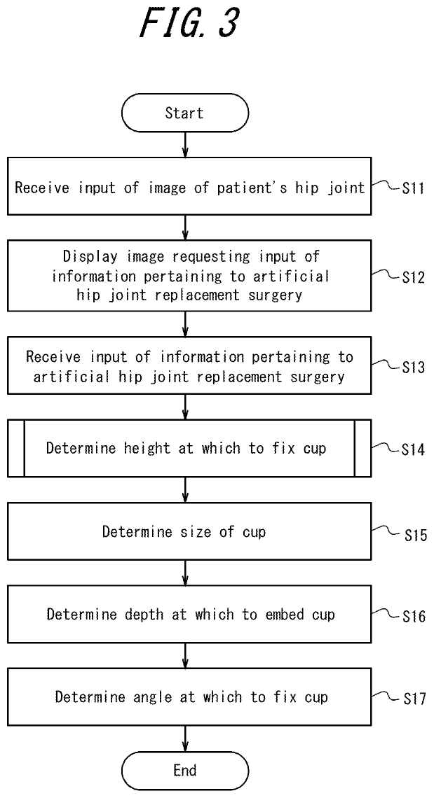

Next, an example of the processing executed by the information processing apparatus 10 for the preoperative plan is described with reference to FIG. 3.

To make a preoperative plan using the information processing apparatus 10, the user of the information processing apparatus 10, such as a doctor, operates the information processing apparatus 10 to input an image of the hip joint of the patient who will receive artificial hip joint replacement surgery. For example, the user performs an operation to input a captured computed tomography (CT) image, x-ray image, or the like of the patient's hip joint to the information processing apparatus 10. The information processing apparatus 10 receives input of the image of the patient's hip joint by the user operation (step S11).

The information processing apparatus 10 displays a screen requesting input of information pertaining to the artificial hip joint replacement surgery on the display 14 (step S12). The information pertaining to the artificial hip joint replacement surgery may include information related to the current condition of the patient's hip joint, required conditions related to the arrangement of the artificial hip joint at the time of the procedure, and the like. The user inputs information pertaining to the artificial hip joint replacement surgery through the input interface 13 in response to the screen displayed on the display 14.

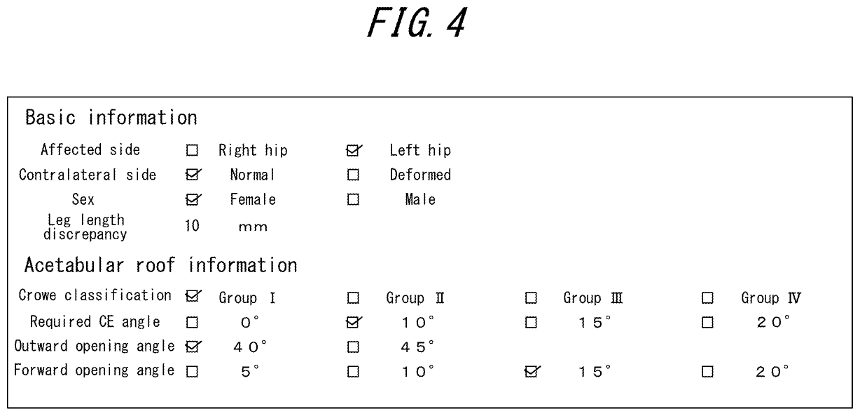

FIG. 4 illustrates an example of information pertaining to the artificial hip joint replacement surgery. In step S12, the information processing apparatus 10 can request input from the user of information pertaining to the artificial hip joint replacement surgery by, for example, displaying information pertaining to the artificial hip joint replacement surgery in checkbox format as illustrated in FIG. 4. The user can input the information pertaining to the artificial hip joint replacement surgery by checking the items, in the checkboxes on the screen displayed by the display 14, applicable to the patient undergoing the procedure. However, the display of information pertaining to the artificial hip joint replacement surgery need not be in checkbox format. It suffices to display the information, pertaining to the artificial hip joint replacement surgery of the patient undergoing the procedure, in any format that allows input, such as radio buttons or a free response format.

In the present embodiment, the user-inputted information pertaining to the artificial hip joint replacement surgery includes basic information and acetabular roof information as illustrated in FIG. 4, for example.

The basic information is information related to the current condition of the patient's hip joint. The basic information includes the affected direction, the condition of the contralateral side, the patient's sex, and the leg length discrepancy.

The affected direction is information indicating which of the patient's left and right hip joints is the affected hip joint to be replaced by the artificial hip joint replacement surgery. In the example in FIG. 4, the affected side is the left hip.

The condition of the contralateral side is information indicating whether the patient's contralateral hip joint (i.e. the opposite side from the affected side) is normal or exhibits deformation. A doctor can, for example, judge whether the condition of the contralateral side is normal or exhibits deformation by examining an image of the hip joint. When the contralateral hip joint is normal, the user checks the box labeled “normal”, for example. When the contralateral hip joint exhibits deformation, the user checks the box labeled “deformed”, for example. In the example in FIG. 4, the contralateral side has been inputted as normal.

The leg length discrepancy is information indicating the difference in length between the patient's left and right legs. In the example in FIG. 4, the leg length discrepancy has been inputted as 10 mm. The leg length discrepancy may be inputted as a numerical value.

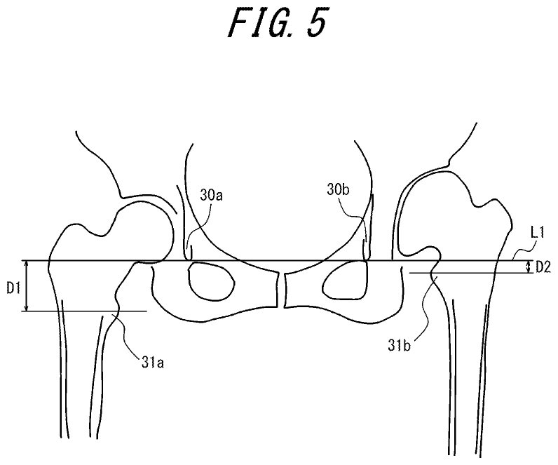

FIG. 5 illustrates the leg length discrepancy, schematically illustrating the pelvis from the front. The leg length discrepancy may, for example, be the difference between the distances from a line L1, connecting the lower edges of left and right teardrops 30a and 30b, to the lesser trochanter apex of the left and right lesser trochanters 31a and 31b, as illustrated in FIG. 5. In the example in FIG. 5, the distance from the line L1 to the lesser trochanter 31a of the right femur is D1, and the distance from the line L1 to the lesser trochanter 31b of the left femur is D2. The leg length discrepancy can, for example, be the difference between D1 and D2. The leg length discrepancy is not, however, limited to this example and may be measured by another method.

In the present disclosure, the direction orthogonal to the line L1 connecting the lower edges of the left and right teardrops 30a and 30b in front view of the patient is referred to as the height direction. Hence, the upward direction in FIG. 5 is the height direction in FIG. 5. The direction towards the center (middle) of the patient's body is referred to as inward, and the direction away from the center as outward in front view of the patient.

Referring again to FIG. 4, the acetabular roof information includes information related to the current condition of the patient's hip joint and required conditions related to the arrangement of the artificial hip joint at the time of the procedure. In the example in FIG. 4, the acetabular roof information includes the Crowe classification of the patient's hip joint, the required CE angle, the outward opening angle, and the forward opening angle.

The Crowe classification of the patient's hip joint is information indicating the classification of the degree of subluxation on the acetabular roof side of the patient's hip joint. The Crowe classification has four groups, from Group I to Group IV. Typically, deformation of the acetabular roof increases from Group I to Group IV. A doctor can, for example, determine the Crowe classification of the patient's hip joint by examining an image of the hip joint. In the example in FIG. 4, the Crowe classification of the patient's hip joint has been inputted as Group I.

The required CE angle is a condition on the center-edge (CE) angle required after the artificial hip joint replacement surgery. In the example in FIG. 4, 10° has been selected as the required CE angle. In other words, a CE angle of at least 10° is required after the artificial hip joint replacement surgery in the example in FIG. 4.

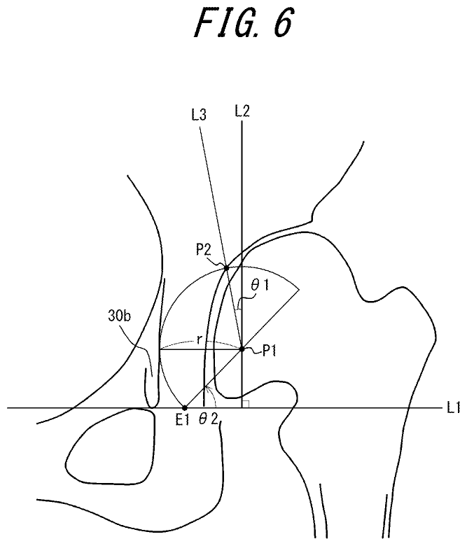

FIG. 6 illustrates the CE angle and the outward opening angle in artificial hip joint replacement surgery. The outward opening angle is described below. As illustrated in FIG. 6, a line L2 in the present disclosure is a line that traverses the center P1 of the cup 25 (cup center) and is perpendicular to the line L1. When the contralateral side is normal, the cup center P1 is the same height as the femoral head center on the contralateral side and is positioned outward, from the outer side of the teardrop 30b, by a distance equal to the radius r of the cup 25. The radius r of the cup 25 may, for example, be the radius of the cup with the highest frequency of use among cups 25 of various sizes or the average radius of cups 25 of various sizes. As illustrated in FIG. 6, a line L3 connects the cup center P1 to a point P2 where the outer circumference of the cup 25 and the acetabulum intersect in front view of the patient when the cup 25 is virtually arranged in a side view. In the example in FIG. 6, a lower edge E1 of the cup in a side view is virtually arranged to touch the line L1. Furthermore, in the example in FIG. 6, the central axis of the cup 25 is virtually arranged to face 45° outward relative to the line L1. The CE angle during artificial hip joint replacement surgery can be defined as the angle of L3 relative to L2. The CE angle can be defined as being positive outward from the line L2 and negative inward from the line L2. In the example in FIG. 6, the CE angle is el and is a negative value.

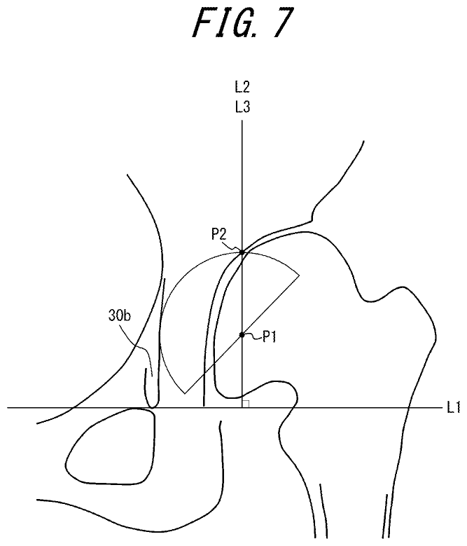

When, for example, the cup 25 is disposed at a higher position (height), the CE angle increases due to the point P2 being positioned further outward along the shape of the acetabulum. For example, FIG. 7 illustrates an example of disposing the cup 25 at a greater height. The lines L2 and L3 overlap in the example in FIG. 7 due to the point P2 being positioned above the cup center P1. The CE angle in this case is 0°. In general, the CE angle tends to increase as deformation of the patient's hip joint is smaller, even when the cup 25 is disposed at the same height. When the acetabulum has suffered high dislocation, the CE angle also generally tends to increase as the cup is disposed higher. The contact area between the cup 25 and the acetabulum increases as the CE angle is larger when the cup 25 is actually disposed in the acetabulum. It therefore becomes easier to fix the cup 25 in the acetabulum.

As the required CE angle in the acetabular roof information illustrated in FIG. 4, the user inputs a condition on the CE angle when the cup 25 is disposed in the hip joint. The user can select from among 0°, 10°, 15°, and 20° as the required CE angle in the example in FIG. 4.

The outward opening angle is the angle to the outside (i.e. outward from the patient) of the opening side of the cup 25 embedded by the artificial hip joint replacement surgery. The outward opening angle is the angle formed by a line segment, i.e. the opening, of the cup 25 in a side view relative to the line L1 connecting the lower edges of the left and right teardrops 30a and 30b. Accordingly, θ2 corresponds to the outward angle in FIG. 6, for example. The patient having the artificial hip joint replacement surgery can move the leg outward more easily when the opening of the cup 25 faces outward rather than directly down. The user can select either 40° or 45° as the outward opening angle in the example in FIG. 4.

The forward opening angle is the forward angle (i.e. to the front of the patient) of the opening side of the cup 25 embedded by the artificial hip joint replacement surgery, i.e. the front opening angle. The patient having the artificial hip joint replacement surgery can move the leg forward more easily when the opening of the cup faces forward. The user can select from among 5°, 10°, 15°, and 20° as the forward opening angle in the example in FIG. 4.

The required CE angle, the outward opening angle, and the forward opening angle, among the acetabular roof information, may be selected without input from the user by using one of the numerical values as a default for each item.

When the user inputs information pertaining to the artificial hip joint replacement surgery, the information processing apparatus 10 receives user input of the information pertaining to the artificial hip joint replacement surgery (step S13). That is, the information processing apparatus 10 receives input of the information pertaining to the artificial hip joint replacement surgery illustrated as an example in FIG. 4.

The information processing apparatus 10 executes processing for the preoperative plan on the basis of an image of the patient's hip joint received as input in step S11 and the information pertaining to the artificial hip joint replacement surgery received as input in step S13. The information processing apparatus 10 makes the preoperative plan by performing step S14 through step S17, for example.

The information processing apparatus 10 first determines the height at which to fix the cup 25, for example, as the preoperative plan (step S14).

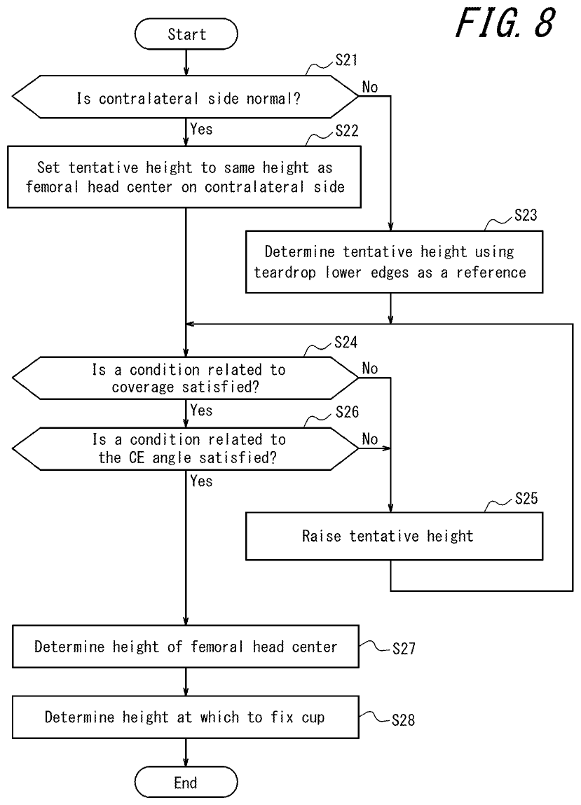

FIG. 8 is a flowchart illustrating an example of processing, executed by the information processing apparatus 10, to determine the height of the cup 25.

The information processing apparatus 10 judges whether the contralateral hip joint of the patient undergoing the procedure is normal (step S21). For example, the information processing apparatus 10 can judge whether the contralateral hip joint of the patient is normal in accordance with user input received in step S13 of FIG. 3. Specifically, when the condition of the contralateral side is inputted as “normal”, the information processing apparatus 10 can judge that the contralateral hip joint is normal (step S21: Yes). Conversely, when the condition of the contralateral side is inputted as “deformed”, the information processing apparatus 10 can judge that the contralateral hip joint is not normal (step S21: No).

When the information processing apparatus 10 judges that the contralateral hip joint is normal (step S21: Yes), the information processing apparatus 10 tentatively sets the height of the femoral head center of the artificial hip joint to the same height as that of the femoral head center on the contralateral side. The femoral head center of the artificial hip joint is, for example, the center of the femoral head ball 22. This tentatively determined height of the femoral head center of the artificial hip joint is referred to as the “tentative height” in the present disclosure. When the contralateral hip joint is judged to be normal (step S21: Yes), the information processing apparatus 10 thus sets the tentative height of the femoral head center of the artificial hip joint to the same height as that of the femoral head center on the contralateral side (step S22).

Conversely, when the contralateral hip joint is judged not to be normal (step S21: No), the information processing apparatus 10 sets the tentative height of the femoral head center of the artificial hip joint using the teardrop lower edges of the patient's acetabula as a reference (step S23). For example, the information processing apparatus 10 assumes that a cup 25, with a radius r and an outward opening angle facing outward 45° relative to the line L1, is disposed so that the lower edge E1 of the cup 25 is in contact with the line L1 in a side view, as illustrated in the example in FIG. 6. The information processing apparatus 10 can, in this case, set the tentative height to the height of the center of the femoral head ball 22 used in correspondence with the cup 25. The method of setting the tentative height when the contralateral hip joint is not normal is not, however, limited to this example. The information processing apparatus 10 may set the tentative height of the femoral head center of the artificial hip joint by another method using the teardrop lower edges as a reference.

After the tentative height of the femoral head center of the artificial hip joint is set in step S22 and step S23, the information processing apparatus 10 judges whether a condition on the coverage of the cup 25 is satisfied at the set tentative height (step S24). The coverage of the cup 25 refers to the proportion of the outer surface of the hemispherical cup 25 that is in contact with the acetabulum when the cup 25 is embedded in the acetabulum. Accordingly, the condition related to coverage of the cup 25 refers to a condition related to the proportion of the outer surface of the cup 25 in contact with the acetabulum. The condition related to coverage of the cup 25 is determined in advance, for example, and stored in the memory 12. The information processing apparatus 10 judges whether the condition, stored in the memory 12, related to the coverage of the cup 25 is satisfied at the set tentative height. For example, a proportion (numerical value) of the outer surface of the hemispherical cup 25 that should be in contact with the acetabulum is stored in the memory 12 as the condition related to coverage. In step S24, the information processing apparatus 10 calculates the coverage of the cup 25 at the set tentative height and judges whether this coverage satisfies the condition related to coverage stored in the memory 12.

As the proportion of the cup 25 in contact with the acetabulum is smaller, the fixed connection between the cup 25 and the acetabulum weakens, making the cup 25 susceptible to detachment. Conversely, as the proportion of the cup 25 in contact with the acetabulum is larger, the fixed connection between the cup 25 and the acetabulum strengthens, making the cup 25 less susceptible to detachment. The cup 25 can therefore be made less susceptible to detachment from the acetabulum to a predetermined degree by setting the coverage of the cup 25 to a predetermined value or higher. The condition related to coverage may be set appropriately in accordance with the degree of reduction in susceptibility of the cup 25 to detachment from the acetabulum.

When it is judged that the condition related to coverage is not satisfied (step S24: No), the information processing apparatus 10 moves the tentative height of the femoral head center to a higher position and sets the new tentative height to the height of the femoral head center of the artificial hip joint after movement (step S25). Processing then transitions to step S24. Due to the shape of a human acetabulum, the contact area between the cup 25 and the acetabulum generally increases as the position is higher than the line L1. The coverage of the cup 25 thus increases as the position is higher than the line L1. Consequently, the condition related to coverage becomes easier to satisfy when the tentative height is raised in step S24.

The movement width when raising the tentative height may be determined appropriately. For example, the movement height may be raised in units of several millimeters. In this case, each time step S25 is executed in the flowchart in FIG. 8, the tentative height is raised by the determined movement width of several millimeters. The movement width when raising the tentative height may be determined by user input. The movement width may be inputted by the user in advance before the start of the flowchart of FIG. 8, for example. In this case, the tentative height is raised by the movement width inputted in advance. The movement width may, for example, be inputted by the user each time step S25 is executed in the flowchart of FIG. 8. In this case, the tentative height is raised by the movement width inputted by the user when step S25 is executed.

When it is judged that the condition related to coverage is satisfied (step S24: Yes), the information processing apparatus 10 judges whether a condition related to the CE angle is satisfied (step S26). The condition related to the CE angle is the condition of “required CE angle” received in step S13 of FIG. 3. Hence, in step S26, the information processing apparatus 10 judges whether the CE angle when the cup 25 is fixed to the acetabulum at the tentative height satisfies the condition of “required CE angle” for which input was received. For example, suppose that 10° was selected as the required CE angle. At this time, the information processing apparatus 10 judges that the condition related to the CE angle is satisfied (step S26: Yes) if the CE angle is at least 10° when the cup 25 is fixed to the acetabulum at the tentative height. The condition related to the CE angle is not limited to being at least 10° and may be at least a predetermined angle that is selected or determined appropriately. Conversely, the information processing apparatus 10 judges that the condition related to the CE angle is not satisfied (step S26: No) if the CE angle is less than 10° when the cup 25 is fixed to the acetabulum at the tentative height.

When it is judged that the condition related to the CE angle is not satisfied (step S26: No), the information processing apparatus 10 moves the tentative height higher and sets the new tentative height to the femoral head center of the artificial hip joint after movement (step S25). Processing then transitions to step S24.

When it is judged that the condition related to the CE angle is satisfied (step S26: Yes), the information processing apparatus 10 sets the height of the femoral head center of the artificial hip joint to the tentative height at the time the condition related to the CE angle was judged to be satisfied (step S27).

The information processing apparatus 10 sets the height at which to fix the cup 25 in accordance with the height of the femoral head center of the artificial hip joint set in step S27 (step S28). In other words, the information processing apparatus 10 sets the height at which to fix the cup 25 to the height at which the cup 25 corresponding to the femoral head ball 22 is disposed when the center of the femoral head ball 22 is at the position, determined in step S27, of the femoral head center of the artificial hip joint.

The processing to determine the height of the cup 25 in step S14 of FIG. 3 is executed in this way. That is, the information processing apparatus 10 can determine the height at which to fix the cup 25 in a preoperative plan for artificial hip joint replacement surgery in accordance with the femoral head center on the contralateral side, the teardrop lower edges, and the CE angle. Specifically, the information processing apparatus 10 determines the tentative height in accordance with the femoral head center on the contralateral side when the patient's contralateral hip joint is normal and in accordance with the height of the teardrop lower edges when the contralateral hip joint is not normal. When conditions related to the CE angle and to coverage are satisfied, the information processing apparatus 10 sets the height of the femoral head center of the artificial hip joint to the tentative height and determines the height at which to fix the cup 25 in accordance with the height of the femoral head center. The information processing apparatus 10 can thus easily determine the appropriate height at which to fix the cup 25.

As described with reference to FIG. 8, the information processing apparatus 10 raises the tentative height of the femoral head center of the femoral head ball 22 when a condition related to coverage is not satisfied and when a condition related to the CE angle is not satisfied and then judges whether the condition related to coverage and the condition related to the CE angle are satisfied at the new tentative height. The height of the femoral head center determined in step S27 therefore becomes the lowest height, among heights that satisfy the condition related to coverage and the condition related to the CE angle, in the movement width interval of the tentative height. Accordingly, the height at which the cup 25 is fixed can easily be set to a height near the original position of the hip joint (i.e. the position at which the hip joint is supposed to be located) while ensuring appropriate conditions of the artificial hip joint.

The information processing apparatus 10 has been described as setting the height of the femoral head center of the artificial hip joint to the tentative height when a condition related to the CE angle and a condition related to coverage are satisfied. The height determination by the information processing apparatus 10 is not, however, restricted to the case of a condition related to the CE angle and a condition related to coverage being satisfied. For example, the information processing apparatus 10 may set the height of the femoral head center of the artificial hip joint to the tentative height when either a condition related to the CE angle or a condition related to coverage is satisfied.

The condition related to coverage has been described as being determined in advance and stored in the memory 12. However, the condition related to coverage is not necessarily stored in advance in the memory 12. For example, in step S12 of FIG. 3, the information processing apparatus 10 may display a condition related to coverage as a portion of the information related to the artificial hip joint replacement surgery and request user input. The user can input the condition related to coverage in this case as a numerical value, for example. Input of the condition related to coverage is received in step S13 of FIG. 3. The information processing apparatus 10 may execute the flowchart of FIG. 8 using the condition related to coverage for which input was received.

Returning to FIG. 3, the information processing apparatus 10 determines the size of the cup 25 (step S15). The information processing apparatus 10 may, for example, determine the size of the cup 25 by selecting an appropriate cup to use for the patient from among cups, stored in the memory 12, that are usable by a doctor.

The information processing apparatus 10 can, for example, determine the size of the cup 25 using the contralateral femoral head diameter of the patient as a reference when the contralateral side is normal. The information processing apparatus 10 can, for example, determine the size of the cup 25 by selecting a cup 25 that has an outer diameter larger than the contralateral femoral head diameter by a certain amount (such as 5 mm).

The information processing apparatus 10 can, for example, determine the size of the cup 25 in accordance with a CT image of the fixing position of the cup 25 at the acetabular roof of the patient when the contralateral side is not normal. The information processing apparatus 10 can, for example, determine the size of the cup 25 by selecting a cup 25 that has an outer diameter allowing fixation to the wall around the acetabular roof in accordance with a CT image inputted by the user.

The information processing apparatus 10 determines the height at which to fix the cup 25 and the depth at which to embed the cup 25 in the acetabulum (step S16). The information processing apparatus 10 can, for example, determine the depth at which to embed the cup 25 using the patient's acetabular fossa surface or outer surface of the teardrop as a reference. The information processing apparatus 10 can, for example, determine to embed the cup 25 so that the outer surface of the cup 25 is in contact with the acetabular fossa surface or the outer surface of the teardrop. In other words, the depth at which to embed the cup 25 is a depth such that the outer surface of the cup 25 is in contact with the acetabular fossa surface or the outer surface of the teardrop.

The information processing apparatus 10 determines the angles at which to fix the cup 25 (step S17). The angles at which to fix the cup 25 include the outward opening angle and the forward opening angle. The information processing apparatus 10 determines to set the angles at which to fix the cup 25 to the outward opening angle and the forward opening angle for which input was received in step S13.

The information processing apparatus 10 can create a preoperative plan using the flowchart of FIG. 3. The information processing apparatus 10 may display the created preoperative plan on the display 14, for example. The information processing apparatus 10 may display information related to the position and angle at which to fix the cup 25 and the size of the cup 25 as the created preoperative plan. A doctor or the like can perform the artificial hip joint replacement surgery in accordance with the displayed preoperative plan.

Various embodiments have been described for a complete and clear disclosure. The appended claims, however, are not limited to the above embodiments and are to be construed as encompassing all of the possible modifications and alternate configurations that a person of ordinary skill in the art could make within the scope of the fundamental features illustrated in this disclosure. The subject matter of the various embodiments may also be freely combined.

For example, a portion of the elements constituting the information processing apparatus 10 according to the above embodiment may be divided up among a plurality of apparatuses to form a system that, overall, achieves the same functions as the information processing apparatus 10.

The information pertaining to the artificial hip joint replacement surgery displayed by the information processing apparatus 10 in step S12 of FIG. 3, for example, has been described in the above embodiment as being the information in FIG. 4. The information pertaining to artificial hip joint replacement surgery is not, however, limited to the example in FIG. 4. The information processing apparatus 10 need not display all of the items in FIG. 4 as the information pertaining to the artificial hip joint replacement surgery. The information processing apparatus 10 may display items not illustrated in FIG. 4 as the information pertaining to the artificial hip joint replacement surgery.

The information processing apparatus 10 may execute different processing in accordance with the Crowe classification of the patient's hip joint, for which input was received in step S13 of FIG. 3. For example, the information processing apparatus 10 may execute different processing in step S14 of FIG. 3 in accordance with the Crowe classification of the hip joint. The information processing apparatus 10 may determine the height at which to fix the cup 25 in step S14 using the teardrop lower edges as a reference when the Crowe classification of the hip joint is Group I or Group IV, for example. The information processing apparatus 10 may determine the height at which to fix the cup 25 in step S14 so that a condition related to the CE angle is satisfied when the Crowe classification of the hip joint is Group II or Group III, for example.

Although this disclosure is based on embodiments and drawings, it is to be noted that various changes and modifications will be apparent to those skilled in the art based on this disclosure. Therefore, such changes and modifications are to be understood as included within the scope of this disclosure. For example, the functions and the like included in the various units, members, and steps may be reordered in any logically consistent way. Furthermore, units and members may be combined into one or divided.

1. An information processing apparatus comprising:
a controller configured to
determine a femoral head center on a contralateral side opposite from an affected hip joint of a patient;
determine a teardrop lower edge of a hip joint of the patient;
determine a center-edge (CE) angle of the affected hip joint of the patient; and
determine a height at which to fix a cup of an artificial hip joint in the patient based on the femoral head center, the teardrop lower edge, and the CE angle.

2. The information processing apparatus of claim 1, wherein the controller is configured to determine a tentative height of the femoral head center of the artificial hip joint, and to determine the height at which to fix the cup based on whether a predetermined condition is satisfied at the tentative height.

3. The information processing apparatus of claim 2, wherein the controller is configured to determine the tentative height based on the femoral head center on the contralateral side when the contralateral side is normal.

4. The information processing apparatus of claim 2, wherein the controller is configured to determine the tentative height based on a height of the teardrop lower edge when the contralateral side is not normal.

5. The information processing apparatus of claim 2, wherein the predetermined condition includes a condition related to the CE angle.

6. The information processing apparatus of claim 5, wherein the condition related to the CE angle is that the CE angle is at least a predetermined angle.

7. The information processing apparatus of claim 6, wherein the predetermined angle is 10°.

8. The information processing apparatus of claim 2, wherein the predetermined condition includes a condition related to coverage of an outer surface of the cup by an acetabulum of the patient when the cup is disposed in the acetabulum.

9. The information processing apparatus of claim 2, wherein the controller is configured to raise the tentative height when the predetermined condition is not satisfied.

10. The information processing apparatus of claim 2, wherein the controller is configured to set a height of the femoral head center of the artificial hip joint to the tentative height when the predetermined condition is satisfied, and to determine the height at which to fix the cup in accordance with the height of the femoral head center of the artificial hip joint.

11. The information processing apparatus of claim 1, wherein the controller is configured to determine a depth at which to embed the cup in an acetabulum of the patient using an acetabular fossa surface of the patient or an outer surface of a teardrop of the patient as a reference.

12. The information processing apparatus of claim 1, wherein the controller is configured to determine a size of the cup using a femoral head diameter of the contralateral side as a reference when the contralateral side is normal.

13. The information processing apparatus of claim 1, wherein the controller is configured to determine a size of the cup based on a computed tomography (CT) image of a fixing position of the cup at an acetabular roof of the patient when the contralateral side is not normal.

14. The information processing apparatus of claim 1, further comprising a display configured to display information determined by the controller.

15. An information processing method executed by an information processing apparatus comprising a controller, the information processing method comprising:
determining a femoral head center on a contralateral side opposite from an affected hip joint of a patient;
determining a teardrop lower edge of a hip joint of the patient;
determining a center-edge (CE) angle of the affected hip joint of the patient; and
determining a height at which to fix a cup of an artificial hip joint in the patient based on the femoral head center, the teardrop lower edge, and the CE angle.

16. A non-transitory computer-readable recording medium storing computer program instructions, that when executed by a computer, perform steps comprising:
determining a femoral head center on a contralateral side opposite from an affected hip joint of a patient;
determining a teardrop lower edge of a hip joint of the patient;
determining a center-edge (CE) angle of the affected hip joint of the patient; and
determining a height at which to fix a cup of an artificial hip joint in the patient based on the femoral head center, the teardrop lower edge, and the CE angle.
